(19) United States
(12) Patent Application Publication
(10) Pub. No.: US 2020/0163897 A1
(43) Pub. Date: May 28, 2020

(54) METHODS OF TREATING LIVER DISEASES ASSOCIATED WITH PORTAL TRACT OR PERIPORTAL INFLAMMATION

(71) Applicant: The Regents of the University of California, Oakland, CA (US)

(72) Inventor: Ranjan Dohil, San Diego, CA (US)

(21) Appl. No.: 16/627,539

(22) PCT Filed: Jun. 25, 2018
(86) PCT No.: PCT/US2018/039254

§ 371 Date: Dec. 30, 2019

Related U.S. Application Data
(60) Provisional application No. 62/528,481, filed on Jul. 4, 2017.

(51) Int. Cl. A61K 9/50; A61K 31/145

(52) U.S. Cl. CPC A61K 9/5094; A61K 31/145

(57) ABSTRACT

The disclosure relates, in general, to treatment of liver disorders associated with portal and periportal inflammation comprising administering compositions comprising cysteamine and/or cystamine compositions.

### CROSS REFERENCE TO RELATED APPLICATIONS

This application claims the priority of U.S. Provisional Application No. 62/528,481, filed Jul. 4, 2017, the disclosures of which are incorporated herein by reference in their entirety.

### FIELD OF THE INVENTION

The invention relates in general to compositions and methods to treat liver disease using cysteamine and cystamine compounds or prodrugs thereof.

### BACKGROUND

Portal tract and peri-portal inflammation is a leading cause/association of liver disease progression. For example, fatty liver disease (or steatohepatitis) is often associated with excessive alcohol intake or obesity, but also has other causes such as metabolic deficiencies including insulin resistance and diabetes. Fatty liver disease results from triglyceride fat accumulation in vacuoles of the liver cells resulting in decreased liver function, and possibly leading to cirrhosis or hepatic cancer. Non-alcoholic fatty liver disease (NAFLD) represents a spectrum of disease occurring in the absence of alcohol abuse and includes non-alcoholic steatohepatitis (NASH). A satisfactory treatment for fatty liver disease, such as NAFLD and NASH is not presently available.

### SUMMARY

The disclosure provides a method of treating a patient suffering from a disease or disorder associated with periportal/portal liver inflammation comprising administering a therapeutically effective amount of a cysteamine salt, cystamine salt, or a prodrug form of either of the foregoing, composition to the patient to obtain a systemic plasma level of about 10-80 μmol of cysteamine in the plasma. In some embodiments, the subject/patient has normal liver function markers but histological inflammation. In another embodiment, the patient/subject has liver disease is selected from the group consisting of NAFLD with pediatric type 2 pattern, autoimmune hepatitis, primary sclerosing cholangitis, primary biliary cholangitis, chronic or acute drug toxicity, biliary atresia, idiopathic neonatal hepatitis syndrome.

The disclosure also provides a method of treating a subject having a liver disease or disorder associated with hepatic periportal inflammation comprising administering cysteamine salt and/or cystamine salt to the subject, wherein inflammation is reduced and/or liver function markers are improved thereby treating the liver disease or disorder. In one embodiment, the liver disease or disorder is selected from the group consisting of NAFLD with pediatric type 2 pattern, autoimmune hepatitis, primary sclerosing cholangitis, primary biliary cholangitis, chronic drug toxicity, biliary atresia, idiopathic neonatal hepatitis syndrome.

In embodiments of either of any of the foregoing, the method further comprises measuring liver function markers selected from AST, GST, ALP and ALT. In still another embodiment of any of the foregoing, the subject is a juvenile. In another embodiment of any of the foregoing, the subject is obese.

The disclosure also provides a method of treating a subject at risk for developing a hepatic disease or disorder associated with periportal/portal inflammation or fibrosis, wherein the subject is at risk for liver disease and has normal liver function, comprising administering cysteamine salt and/or cystamine salt to the subject. In one embodiment, the subject comprises normal liver function markers or abnormal liver function markers, wherein the markers of liver function are selected from the group consisting of alanine aminotransferase (ALT), alkaline phosphatase (ALP), aspartate aminotransferase (AST), gamma-glutamyl transpeptidase (GGT) and triglycerides. In one embodiment, ALT or AST levels above age-established normal values are considered abnormal (e.g., an ALT above 30 can be considered abnormal in children at certain agents). In a further embodiment, an ALT level is normal or elevated and wherein an elevated level of about 60 or higher units/liter is indicative of fatty liver disease. In another further embodiment, the ALP level is normal or elevated and wherein an elevated level of about 150 or higher units/liter is indicative of fatty liver disease. In still yet another further or alternative embodiment, an AST level is normal or elevated and wherein an elevated level of about 40 or higher units/liter is indicative of fatty liver disease. In still another further or alternative embodiment, a GGT level is normal or elevated and wherein an elevated level of about 50 or higher units/liter is indicative of fatty liver disease. In still another embodiment, a triglyceride level above 150 mg/dL and/or high LDL level is indicative of fatty liver disease. In another embodiment, the subject's BMI is above the 97^{th} percentile for the subject's age and the subject weighs less than or equal to 65 kg. In another embodiment, the subject comprises a BMI above 97^{th} percentile for age and weighs less than 65 kg and has triglyceride levels associated with NASH. In one embodiment, the cysteamine and/or cystamine salt composition is a delayed release formulation. In yet another embodiment, the cysteamine and/or cystamine salt composition is an extended release formulation. In still other embodiments, the cysteamine and/or cystamine salt composition is a delayed and extended release formulation. In yet another embodiment, the cysteamine and/or cystamine salt composition is an enterically coated composition. In another embodiment, the cysteamine salt is cysteamine bitartrate. In still another or further embodiment, the cysteamine salt compositions comprise granules or tablets. In another or alternative embodiment, the cysteamine salt composition comprises (i) a core particle comprising a mixture of cysteamine bitartrate and a binder, and (ii) an enteric membrane surrounding the core particle; wherein the beads have a distribution of particle sizes in a range of about 0.7 mm to about 2.8 mm; wherein the enteric membrane begins to dissolve within a pH range of about 4.5 to about 6.5; wherein the enteric membrane is present in an amount in a range of about 25% to about 35% by weight, based on the weight of the core particles; and wherein the pharmaceutical dosage form, upon administration in a capsule to fasted healthy normal subjects at 600 mg free cysteamine base, provides: (a) a mean Cₘₐₓ upon oral dosing in a range of 2.3±0.6 mg/L or in a range of 80% to 125% thereof; (b) a mean AUC (0-∞) upon oral dosing in a range of 0.84±0.19 min*mg/L/mg or in a range of 80% to 125% thereof; and (c) a plasma cysteamine level of 10-80 μmol. In yet another or alternative embodiment, the cysteamine salt composition comprises (i) a core tablet comprising a mixture of cysteamine bitartrate and a binder, and (ii) an enteric membrane surrounding the tablet, wherein the thickness of the enteric coating increases from 70 μm to 115 μm relative to the cysteamine base dose from 50 mg to 300 mg, and/or wherein the enteric coating is present in an amount in a range of about 9 to about 15% by weight of the core tablet and wherein upon delivery to a fasted healthy normal subject at 600 mg free cysteamine base the dose provides a plasma cysteamine level of 10-80 μm. In still another embodiment, the cystamine salt composition is an enterically coated composition. In a further or alternative embodiment, the cystamine salt compositions comprise granules or tablets. In yet another embodiment, the cystamine salt composition comprises (i) a core particle comprising a mixture of cystamine and a binder, and (ii) an enteric membrane surrounding the core particle; wherein the beads have a distribution of particle sizes in a range of about 0.7 mm to about 2.8 mm; wherein the enteric membrane begins to dissolve within a pH range of about 4.5 to about 6.5; wherein the enteric membrane is present in an amount in a range of about 25% to about 35% by weight, based on the weight of the core particles; and wherein the pharmaceutical dosage form, upon administration in a capsule to fasted healthy normal subjects at 600 mg free cystamine base, provides: (a) a mean Cₘₐₓ upon oral dosing in a range of 2.3±0.6 mg/L or in a range of 80% to 125% thereof; (b) a mean AUC (0-∞) upon oral dosing in a range of 0.84±0.19 min*mg/L/mg or in a range of 80% to 125% thereof; and (c) a plasma cysteamine level of 10-80 μmol. In still another embodiment, the cystamine salt composition comprises (i) a core tablet comprising a mixture of cystamine and a binder, and (ii) an enteric membrane surrounding the core tablet, wherein the thickness of the enteric coating increases from 60 μm to 130 μm relative to the cystamine dose from 50 mg to 300 mg, and/or wherein the enteric coating is present in an amount in a range of about 9 to about 15% by weight of the core tablet and wherein upon delivery to a fasted healthy normal subject at 600 mg cystamine the dose provides a plasma cysteamine level of 10-80 μm.

### BRIEF DESCRIPTION OF THE DRAWINGS

FIG. 1 shows mean plasma cysteamine levels following intra-duodenal cysteamine (20 mg/kg body weight). Samples were taken from the portal vein (PV) and jugular vena cava (JVC) in rats that were either pre-treated with or naïve to cysteamine therapy.

### DETAILED DESCRIPTION

As used herein and in the appended claims, the singular forms “a,” “and” and “the” include plural referents unless the context clearly dictates otherwise. Thus, for example, reference to “a derivative” includes a plurality of such derivatives and reference to “a subject” includes reference to one or more subjects and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

The term “about” as used herein can allow for a degree of variability in a value or range, for example, within 10%, within 5%, or within 1% of a stated value or of a stated limit of a range. When a range or a list of sequential values is given, unless otherwise specified any value within the range or any value between the given sequential values is also disclosed.

The term “substantially” as used herein refers to a majority of, or mostly, as in at least about 51%, 60%, 70%, 80%, 90%, 95%, 96%, 97%, 98%, 99%, 99.5%, 99.9%, 99.99%, or at least about 99.999% or more.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

Any publications discussed above and throughout the text are provided solely for their disclosure prior to the filing date of the present application. Nothing herein is to be construed as an admission that the inventors are not entitled to antedate such disclosure by virtue of prior disclosure.

Non-alcoholic fatty liver disease (NAFLD) is the most common cause of chronic liver disease in children. NAFLD includes a range of disease states from benign steatosis to non-alcoholic steatohepatitis (NASH). The disease often leads to cirrhosis of the liver resulting in the need for liver transplantation as well as causing other problems including, but not limited to, metabolic and cardiovascular disease. Although the pathogenesis of NAFLD is still unclear it is likely that insulin resistance, increased oxidative stress and lipid peroxidation play roles. Levels of intracellular glutathione, which protects against oxidative stress, are low in NAFLD subjects.

Two distinct histological forms of NASH have been described. Type 1 NASH occurs in adults and some children and is characterized by steatosis, lobular inflammation, ballooning degeneration and perisinusoidal fibrosis. Type 2 NASH, found most commonly in children, is characterized by steatosis, portal inflammation, and portal fibrosis. Schwimmer et al. (Hepatgology, 42(3):641-649, 2005; incorporated herein by reference) described various criteria and biomarkers used to differentiate NASH Type 1 from NASH Type 2. In particular, Schwimmer et al. discloses that subjects with NASH Type 1 had higher AST, ALT and triglyceride levels compared to patients with NASH Type 2. However, the strongest factor demonstrating a difference in the two types of NASH is observed upon histological examination. As stated above, Type 1 NASH demonstrates a prevalent lobular inflammation in the liver in contrast with a prevalent portal inflammation in Type 2 NASH. Thus, the disclosure contemplates that one of the key differentiating factors that can be used in the methods disclosed herein is identifying, by histological examination, the presence of Type 1 NASH vs. Type 2 NASH.

As mentioned, NAFLD represents a spectrum of disease occurring in the absence of alcohol abuse. It is characterized by the presence of steatosis (fat in the liver) and may represent a hepatic manifestation of the metabolic syndrome (including obesity, diabetes and hypertriglyceridemia). NAFLD is linked to insulin resistance, it causes liver disease in adults and children and may ultimately lead to cirrhosis (Skelly et al., J. Hepatol., 35:195-9, 2001; Chitturi et al., Hepatology, 35(2):373-9, 2002). The severity of NAFLD ranges from the relatively benign isolated predominantly macrovesicular steatosis (i.e., nonalcoholic fatty liver or NAFL) to non-alcoholic steatohepatitis (NASH) (Angulo et al., J Gastroenterol. Hepatol., 17 Suppl: S186-90, 2002). NASH is characterized by the histologic presence of steatosis, cytological ballooning, scattered inflammation and pericellular fibrosis (Contos et al., Adv. Anat. Pathol., 9:37-51, 2002). Hepatic fibrosis resulting from NASH may progress to cirrhosis of the liver or liver failure, and in some instances may lead to hepatocellular carcinoma.

The degree of insulin resistance (and hyperinsulinemia) correlates with the severity of NAFLD, being more pronounced in patients with NASH than with simple fatty liver (Sanyal et al., Gastroenterology, 120(5):1183-92, 2001). As a result, insulin-mediated suppression of lipolysis occurs and levels of circulating fatty acids increase. Two factors associated with NASH include insulin resistance and increased delivery of free fatty acids to the liver. Insulin blocks mitochondrial fatty acid oxidation. The increased generation of free fatty acids for hepatic re-esterification and oxidation results in accumulation of intrahepatic fat and increases the liver's vulnerability to secondary insults.

Glutathione (gammaglutamyl-cysteinyl-glycine; GSH) is a major endogenous antioxidant and its depletion is implicated in the development of hepatocellular injury (Wu et al., J. Nutr., 134(3):489-92, 2004). One such injury is acetaminophen poisoning, where reduced GSH levels become depleted in an attempt to conjugate and inactivate the hepatotoxic metabolite of the drug. After a toxic dose of acetaminophen, excess metabolite (N-acetyl-benzoquinoneimine) covalently binds to hepatic proteins and enzymes resulting in liver damage (Wu et al., supra; Prescott et al., Annu Rev Pharmacol Toxicol., 23:87-101, 1983). Increased glutathione levels appears therefore to have some protective effects through the reduction of reactive oxygen species (ROS). Glutathione itself does not easily enter into cells, even when given in large amounts. However, glutathione precursors do enter into cells and some GSH precursors such as N-acetylcysteine have been shown to be effective in the treatment of conditions such as acetaminophen toxicity by slowing or preventing GSH depletion (Prescott et al., supra). Examples of GSH precursors include cysteine, N-acetylcysteine, methionine and other sulphur-containing compounds such as cysteamine (Prescott et al., J. Int. Med. Res., 4(4 Suppl): 112-7, 1976).

Cysteine is a major limiting factor for GSH synthesis and that factors (e.g., insulin and growth factors) that stimulate cysteine uptake by cells generally result in increased intracellular GSH levels (Lyons et al., Proc. Natl. Acad. Sci. U.S.A., 97(10):5071-6, 2000; Lu S C., Curr. Top. Cell Regul., 36:95-11, 2000).

N-acetylcysteine has been administered to patients with NASH. In reports from Turkey, obese individuals with NASH treated with N-acetylcysteine for 4-12 weeks exhibited an improvement in aminotransferase levels and gamma-GT even though there was no reported change in subject body mass index (Pamuk et al., J. Gastroenterol. Hepatol., 18(10):1220-1, 2003).

Studies in mice and humans showed cysteamine to be effective in preventing acetaminophen-induced hepatocellular injury (Prescott et al., Lancet, 2(7778):652, 1972; Prescott et al., Br. Med. J., 1(6116):856-7, 1978; Mitchell et al., Clin. Pharmacol. Ther., 16(4):676-84, 1974). Cystamine and cysteamine have been reported to reduce liver cell necrosis induced by several hepatotoxins. (Toxicol. Appl. Pharmacol., 48(2):221-8, 1979). Cystamine has been shown to ameliorate liver fibrosis induced by carbon tetrachloride via inhibition of tissue transglutaminase (Qiu et al., World J. Gastroenterol., 13:4328-32, 2007).

The prevalence of NAFLD in children is unknown because of the requirement of histologic analysis of liver in order to confirm the diagnosis (Schwimmer et al., Pediatrics, 118(4):1388-93, 2006). However, estimates of prevalence can be inferred from pediatric obesity data using hepatic ultra-sonography and elevated serum transaminase levels and the knowledge that 85% of children with NAFLD are obese. Data from the National Health and Nutrition Examination Survey has revealed a threefold rise in the prevalence of childhood and adolescent obesity over the past 35 years; data from 2000 suggests that 14-16% children between 6-19 years of age are obese with a BMI>95% (Fishbein et al., J. Pediatr. Gastroenterol. Nutr., 36(1):54-61, 2003), and also that fact that 85% of children with NAFLD are obese.

The exact mechanism by which NAFLD develops into NASH remains unclear. Because insulin resistance is associated with both NAFLD and NASH, it is postulated that other additional factors are also required for NASH to arise. This is referred to as the “two-hit” hypothesis (Day C P., Best Pract. Res. Clin. Gastroenterol., 16(5):663-78, 2002) and involves, firstly, an accumulation of fat within the liver and, secondly, the presence of large amounts of free radicals with increased oxidative stress. Macrovesicular steatosis represents hepatic accumulation of triglycerides, and this in turn is due to an imbalance between the delivery and utilization of free fatty acids to the liver. During periods of increased calorie intake, triglyceride will accumulate and act as a reserve energy source. When dietary calories are insufficient, stored triglycerides (in adipose) undergo lipolysis and fatty acids are released into the circulation and are taken up by the liver. Oxidation of fatty acids will yield energy for utilization. Treatment of NASH currently revolves around the reduction of the two main pathogenetic factors, namely, fat accumulation within the liver and excessive accumulation of free radicals causing oxidative stress. Fat accumulation is diminished by reducing fat intake as well as increasing caloric expenditure. One therapeutic approach is sustained and steady weight loss. Although not definitively proven, a >10% loss in body weight has been shown in some cases to reduce hepatic fat accumulation, normalize liver transaminases and improve hepatic inflammation and fibrosis (Ueno et al., J, Hepatol., 27(1):103-7, 1997; Vajro et al., J, Pediatr., 125(2):239-41, 1994; Franzese et al., Dig. Dis. Sci., 42(7):1428-32, 1997).

Reduction of oxidative stress through treatment with antioxidants has also been shown to be effective in some studies. For example, obese children who had steatosis were treated with vitamin E (400-1000 IU/day) for 4-10 months (Lavine, J. Pediatr., 136(6):734-8, 2000). Despite any significant change in BMI, the mean ALT levels decreased from 175±106 IU/L to 40±26 IU/L (P<0.01) and mean AST levels decreased from 104±61 IU/L to 33±11 IU/L (P<0.002). Hepatic transaminases increased in those patients who elected to discontinue vitamin E therapy. An adult study using vitamin E for one year demonstrated similar reduction of hepatic transaminases as well as the fibrosis marker TGF levels (Hasegawa et al., Aliment. Pharmacol. Ther., 15(10):1667-72, 2001).

Steatosis also may develop into steatohepatitis through oxidative stress due to reactive oxygen species (ROS) and decreased anti-oxidant defense (Sanyal et al., Gastroenterology, 120(5):1183-92, 2001). ROS can be generated in the liver through several pathways including mitochondria, peroxisomes, cytochrome P450, NADPH oxidase and lipooxygenase (Sanyal et al., Nat. Clin. Pract. Gastroenterol. Hepatol., 2(1):46-53, 2005). Insulin resistance and hyperinsulinism has been shown to increase hepatic oxidative stress and lipid peroxidation through increased hepatic CYP2EI activity (Robertson et al., Am. J. Physiol. Gastrointest. Liver Physiol., 281(5): G1135-9, 2001; Leclercq et al., J. Clin. Invest., 105 (8): 1067-75, 2000).

Currently, much of what is understood of the pathogenesis of NAFLD has arisen from animal studies. A number of mouse models that exhibit steatosis/steatohepatitis exist and include genetically altered leptin-deficient (ob/ob) or leptin resistant (db/db) and the dietary methionine/choline deficient (MCD) model. Studies comparing male and female rats of varying strains (Wistar, Sprague-Dawley, Long-Evans) with a mouse strain (C57BL/6) as models for NASH have been undertaken. When these animals were fed for 4 weeks with an MCD diet, although ALT elevation and steatosis were more noticeable in the Wistar rat, the overall histologic changes in the liver of the mice were more consistent with changes due to NASH. More recently the use of supra-nutritional diets in animals has resulted in a NAFLD model that physiologically more resembles the human phenotype. The medical conditions most commonly associated with NAFLD are obesity, Type II diabetes and dyslipidemia. These conditions can be induced by feeding mice and rats with high fat or sucrose diets. Rats fed with a >70% fat-rich diet for 3 weeks developed pan-lobular steatosis, patchy inflammation, enhanced oxidative stress, and increased plasma insulin concentrations suggesting insulin resistance. NASH mice have been induced through intragastric overfeeding. Mice fed up to 85% in excess of their standard intake for 9 weeks became obese with 71% increase in final body weight; they demonstrated increase white adipose tissue, hyperglycemia, hyperinsulinemia, hyperleptinemia, glucose intolerance and insulin resistance. Of these mice 46% developed increased ALT (121±27 vs 13±1 U/L) as well as histologic features suggestive of NASH. The livers of the overfed mice were about twice as large as expected, beige in color with microscopic evidence of lipid droplets, cytoplasmic vacuoles and clusters of inflammation.

Mouse models of NASH can be used to study various therapies. Mouse models are created through specific diets (methionine choline deficient, MCD) or intragastric overfeeding. These mice develop serologic and histologic features of NASH. NASH mice are useful in screening and measuring the effects cysteamine on NASH related disease and disorders. For example, the effect of treatment can be measured by separating the NASH mice into a control group where animals will continue to receive MCD diet only and four other treatment groups where mice will receive (i) MCD diet as well as anti-oxidant therapy, (ii) cysteamine 50 mg/kg/day, (iii) cysteamine 100 mg/kg/day and (iv) sAME.

As mentioned above, NASH is a disease subset falling under the umbrella of NAFLD and is characterized by various biomarkers and histological examination. Type 1 and Type 2 NASH have some distinct biomarker and histological characteristics that differentiate the two types, while at the same time having certain others that overlap between the two. Type 1 and Type 2 NASH are typically identified in juvenile patients.

To date, there is no single effective treatment for NASH either in the pediatric or adult populations. Possible treatment strategies have included improving insulin sensitivity and to reduce the damage caused by oxidative stress.

The aminothiol agent cysteamine has been approved for the treatment of cystinosis and is commercially available in a twice daily enteric-coated microsphere delayed and prolonged-release form (Procysbi®, manufactured by Horizon Pharmaceuticals).

Cysteamine is a small aminothiol molecule that is easily transported across cellular membranes. Cysteamine markedly reduces intralysosomal cysteine accumulation and is currently approved as a treatment for cystinosis. Cysteamine can increase the cellular thiol and free thiol tripeptide glutathione pool, and thus modulate reactive oxygen species (ROS) scavenging, and decreased lipoperoxidation and glutathione peroxidase activity. Furthermore, cysteamine also increases adiponectin levels. Cysteamine exists in various salt forms the most common of which are —HCl and -bitartrate.

Cystamine is an organic disulfide. Cystamine is generally handled as the dihydrochloride salt, C₄H₁₂N₂S₂.2HCl. Cystamine in the body is reduced to cysteamine and RS-cysteamine mixed disulphide. Accordingly, both cystamine and cysteamine can be used in the methods described herein. For example, cystamine can be used to provide two cysteamine molecules per cystamine, thus providing less frequent or smaller dosing to accomplish similar results to that with cysteamine.

As used herein “cysteamine” and “cystamine” include their pharmaceutically acceptable salts.

Cysteamine and/or cystamine are attractive candidates for the treatment of NASH, as it reacts with cystine to produce cysteine, which can further be metabolized into glutathione, a potent endogenous antioxidant. Cysteamine is a precursor to the protein glutathione (GSH) precursor. Moreover, cysteamine is currently FDA approved for use in the treatment of cystinosis, an intra-lysosomal cystine storage disorder. In cystinosis, cysteamine acts by converting cystine to cysteine and cysteine-cysteamine mixed disulfide which are then both able to leave the lysosome through the cysteine and lysine transporters respectively (Gahl et al., N Engl J Med, 347(2):111-21, 2002). Within the cytosol the mixed disulfide can be reduced by its reaction with glutathione and the cysteine released can be used for further GSH syntheses. The synthesis of GSH from cysteine is catalyzed by two enzymes, gamma-glutamylcysteine synthetase and GSH synthetase. This pathway occurs in almost all cell types, with the liver being the major producer and exporter of GSH. The reduced cysteine-cysteamine mixed disulfide will also release cysteamine, which, in theory, is then able to re-enter the lysosome, bind more cystine and repeat the process (Dohil et al., J Pediatr, 148(6):764-9, 2006). In a recent study in children with cystinosis, enteral administration of cysteamine resulted in increased cysteamine absorption, which subsequently caused prolonged efficacy in the lowering of leukocyte cystine levels (Id.). This may have been due to “re-cycling” of cysteamine when adequate amounts of drug reached the lysosome. If cysteamine acts in this fashion, then GSH production may also be significantly enhanced.

Cysteamine is a potent gastric acid-secretagogue that has been used in laboratory animals to induce duodenal ulceration. Studies in humans and animals have shown that cysteamine-induced gastric acid hypersecretion is most likely mediated through hypergastrinemia. In previous studies performed in children with cystinosis who suffered regular upper gastrointestinal symptoms, a single oral dose of cysteamine (11-23 mg/kg) was shown to cause hypergastrinemia and a 2 to 3-fold rise in gastric acid-hypersecretion, and a 50% rise in serum gastrin levels. Symptoms suffered by these individuals included abdominal pain, heartburn, nausea, vomiting, and anorexia. Dohil et al. (see, e.g., U.S. Pat. No. 8,026,284 and published International Publication No. WO 2007/089670, both claiming priority to U.S. Provisional Patent application No. 60/762,715, filed Jan. 26, 2006; all of which are incorporated by reference herein in their entirety) showed that cysteamine induced hypergastrinemia arises, in part, as a local effect on the gastric antral-predominant G-cells in susceptible individuals. The data also suggest that this is also a systemic effect of gastrin release by cysteamine. Depending on the route of administration, plasma gastrin levels usually peak after intragastric delivery within 30 minutes whereas the plasma cysteamine levels peak later.

International Publication No. WO 2007/089670 demonstrates that delivery of cysteamine to the small intestine reduces gastric distress and ulceration, increases Cₘₐₓ and increases AUC. Delivery of cysteamine into the small intestine is useful due to improved absorption rates from the small intestine, and/or less cysteamine undergoing hepatic first pass elimination when absorbed through the small intestine. A decrease in leukocyte cystine was observed within an hour of treatment.

In addition, sulfhydryl (SH) compounds such as cysteamine, cystamine, and glutathione are among the most important and active intracellular antioxidants. Cysteamine protects animals against bone marrow and gastrointestinal radiation syndromes. The rationale for the importance of SH compounds is further supported by observations in mitotic cells. These are the most sensitive to radiation injury in terms of cell reproductive death and are noted to have the lowest level of SH compounds. Conversely, S-phase cells, which are the most resistant to radiation injury using the same criteria, have demonstrated the highest levels of inherent SH compounds. In addition, when mitotic cells were treated with cysteamine, they became very resistant to radiation. It has also been noted that cysteamine may directly protect cells against induced mutations. The protection is thought to result from scavenging of free radicals, either directly or via release of protein-bound GSH.

Although not wishing to be bound by a specific theory, it is contemplated that cysteamine may increase the cellular thiol pool, increase intracellular glutathione and thereby reduce oxidative stress and lipoperoxidation in NAFLD. Cysteamine may also reduce insulin resistance by increasing production and release of the adipocytokine adiponectin. Cysteamine has also been shown to diminish fibrosis through its effect on TGF-beta and transglutaminases.

Two previous studies of children with NASH have shown that treatment with cysteamine for 6 and 12 months resulted in a statistically significant improvement in blood liver inflammation markers including alanine aminotransferase (ALT), aspartate aminotransferase (AST) and gamma glutamyl transferase (GGT). The improvements in the markers of liver inflammation were sustained for 6 months after discontinuing therapy. In the placebo controlled 12 month study group, the primary endpoint of improved histology was not achieved, however, a post-hoc analysis suggested that children <65 kg body weight had a 4-fold chance of improvement compared with the placebo group. These patients received a relatively higher dose of cysteamine per/kg body weight compared with the heavier children in the study. This study did not specify which histological type of NASH would be likely to respond to cysteamine therapy, if at all.

A pilot trial by Dohil et al. in 11 children with biopsy-confirmed NAFLD received enteric-coated (EC) cysteamine bitartrate orally for 24 weeks. This therapy resulted in statistically significant reductions in mean serum levels of ALT, aspartate aminotransferase (AST), total adiponectin, leptin, and cytokeratin-18 fragments, but without a concomitant reduction in body mass index. Seven out of 11 subjects reached the primary endpoints (of at least 50% reduction in ALT). The reduction in mean ALT and AST levels persisted 16 weeks after treatment ended. However, Schwimmer et al. (Hepatology, 62(6):1398A-1399A, December 2015) indicated that after one year of treatment with delayed released cysteamine bitartrate the therapy was safe, but did not improve liver histology. Although there was substantial and rapid improvements in liver enzymes with the delayed release cysteamine bitartrate.

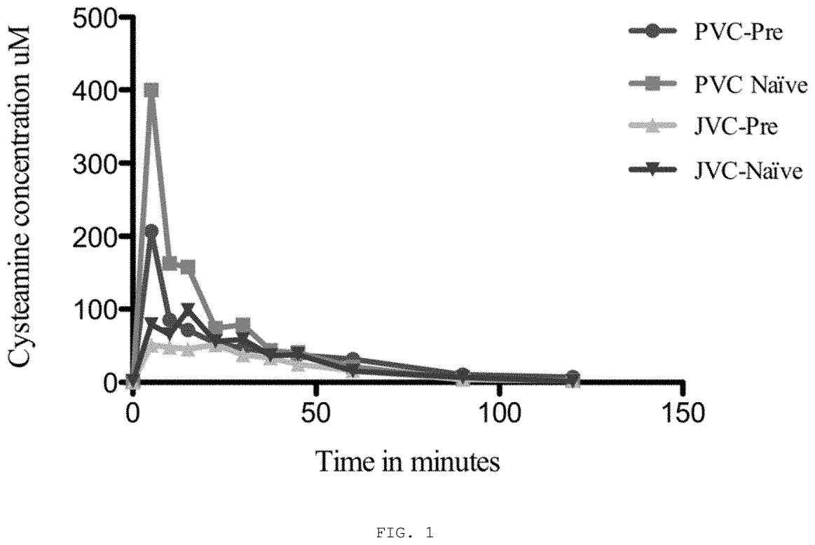

A pharmacokinetics study of cysteamine delivered intraduodenally to rats showed that the plasma cysteamine Cₘₐₓ and AUC measurements were 3 times and 2 times, respectively, higher when drug levels were measured directly from the portal vein compared with the jugular vena cava (JVC) (see, FIG. 1 and Table 1). The portal vein delivers blood, and therefore drug, directly to the liver where it diffuses into the periportal region. The concentration of drug in the periportal region is therefore at a maximum level and is higher than the drug concentration in the hepatic lobular region (which drains into the hepatic central veins and then into the JVC). This is possible because as blood, and therefore drug, traverses through the hepatic lobule away from the portal vein its contents will undergo some degree of first-pass metabolism. It is postulated here, that patients with type 2 pediatric NASH, receiving a greater drug exposure at the site of maximal disease (i.e., periportal region; recall Type 2 NASH is characterized by portal inflammation), are more likely to respond to drug therapy than children with type 1 NASH. If those with type 1 NASH are to respond then they will require a higher dose of cysteamine (e.g., above 10 mg/kg body weight) as by the time drug arrives at the site of maximal disease (i.e., lobular region; recall Type 1 NASH has lobular inflammation common in adult subjects) the local drug concentrations will already have been diminished by hepatic first-pass metabolism which is estimated to be ˜40% for cysteamine.

TABLE 1
Mean pharmacokinetic parameters following intra-duodenal cysteamine
(20 mg/kg) delivery in rats that were naïve and pre-treated
with cysteamine. The ratio AUC_{JVC}/AUC_{PVC} would suggest a
hepatic first-pass effect of about 40%.

| | PORTAL VEIN | | JUGULAR VENA CAVA | |
|---|---|---|---|---|
| | Naive | Pre-treated | Naive | Pre-treated |
| AUC | 6433 | 5171 | 3761 | 2800 |
| (min*uM) | (±1603) | (±1104) | (±813) | (±383) |
| | P = 0.16 | | P = 0.03* | |
| Cₘₐₓ | 281.4 | 147.6 | 81.9 | 53.4 |
| (uM) | (±142.7) | (±72.2) | (±22.9) | (±7.2) |

| | | |
|---|---|---|
| | P = 0.08 | P = 0.02* |
| Tₘₐₓ | 5-10 | 5-22.5 |
| (minutes) | | |
| AUC_{JVC}/ | 54% | 58% |
| AUC_{PVC} | | |

*denotes statistical significance between naïve and pre-treated groups.

The high portal tract and periportal concentrations of cysteamine within the liver as well as the drugs pleotropic actions would make it an important potential agent in the treatment of chronic liver disorders which are characterized by portal tract, periportal and interface hepatitis and fibrosis. Examples of such disorders would include, but are not limited to, NAFLD with pediatric type 2 pattern, autoimmune hepatitis, primary sclerosing cholangitis, primary biliary cholangitis, chronic drug toxicity, biliary atresia, and idiopathic neonatal hepatitis syndrome. Moreover, the disclosure contemplates that treating early portal inflammation in type-2 presenting subjects, will reduce the advancement of liver disease to later stage or adult type-1 liver disease.

This disclosure identifies patient populations that can benefit from cysteamine therapy, particularly subjects with periportal/portal hepatic inflammation. More particularly, subjects with early hepatic inflammation and more particularly juvenile patients, whose progression from NAFLD to NASH may be treated and/or prevented.

The disclosure is not limited with respect to a specific cysteamine or cystamine salt or ester or derivative or any particular formulation (e.g., immediately release, delayed release, extended release, delayed and extended release, immediate and extended release etc.); the compositions of the disclosure can contain any cysteamine or cystamine, cysteamine or cystamine derivative, or combination of cysteamine and cystamine in any formulation. The active agents in the composition, i.e., cysteamine or cystamine, may be administered in the form of a pharmacologically acceptable salt, ester, amide, prodrug or analog or as a combination thereof. Salts, esters, amides, prodrugs and analogs of the active agents may be prepared using standard procedures known to those skilled in the art of synthetic organic chemistry and described, for example, by J. March, “Advanced Organic Chemistry: Reactions, Mechanisms and Structure,” 4th Ed. (New York: Wiley-Interscience, 1992). For example, basic addition salts are prepared from the neutral drug using conventional means, involving reaction of one or more of the active agent's free hydroxyl groups with a suitable base. Generally, the neutral form of the drug is dissolved in a polar organic solvent such as methanol or ethanol and the base is added thereto. The resulting salt either precipitates or may be brought out of solution by addition of a less polar solvent. Suitable bases for forming basic addition salts include, but are not limited to, inorganic bases such as sodium hydroxide, potassium hydroxide, ammonium hydroxide, calcium hydroxide, trimethylamine, or the like. Preparation of esters involves functionalization of hydroxyl groups which may be present within the molecular structure of the drug. The esters are typically acyl-substituted derivatives of free alcohol groups, i.e., moieties which are derived from carboxylic acids of the formula R—COOH where R is alkyl, and typically is lower alkyl. Esters can be reconverted to the free acids, if desired, by using conventional hydrogenolysis or hydrolysis procedures. Preparation of amides and prodrugs can be carried out in an analogous manner. Other derivatives and analogs of the active agents may be prepared using standard techniques known to those skilled in the art of synthetic organic chemistry, or may be deduced by reference to the pertinent literature.

In determining whether a patient has NAFLD, NASH and/or the type of NASH (e.g., Type 1 or Type 2), various markers are measured. The diagnosis of steatosis is typically made when lipid deposition is visible in more than 5% of hepatocytes. NASH is diagnosed when, in addition to hepatic steatosis, both inflammatory infiltrates as well as ballooning and liver cell injury are present. The NAFLD Activity Score (NAS) was developed to provide a numerical score for patients who most likely have NASH. Accordingly, the NAS is the sum of the separate scores for steatosis (0-3), hepatocellular ballooning (0-2) and lobular inflammation (0-3), with the majority of patients with NASH having a NAS score of ≥5 (Kleiner et al., Hepatology, 41(6):1313-1321, 2005). As will be apparent from the scoring matrix above, subjects with periportal inflammation and low to no lobular inflammation will have a lower score compared to those with lobular inflammation. Such patients may thus not be diagnosed with NASH although they would benefit from therapy due to the periportal inflammation of the liver.

In addition, various studies have shown that cytokeratin 18 is a useful indicator of inflammation in NASH, due to cytokeratin 18's release from hepatocytes undergoing apoptosis. Normal cytokeratin 18 levels are typically characterized as being less than 200 units per liter. In contrast, subjects with liver disease, including NALFD and NASH have a statistically significant elevation in cytokeratin 18 (e.g., above 200 U/L; ˜200-300 U/L). Moreover, cytokeratin 18 levels can be used as a marker to determine whether a treatment is being effective. For example, a reduction in cytokeratin 18 levels of greater than 10% (e.g., 20-30%, 30-40%, 40-50%, 50-60%, 60-70%, 70-80% or 90-100%, or any integer between 10%-100%) is indicative that the therapy is having a beneficial effect. Other markers include commonly used liver function tests including measuring one or more of, for example, serum alanine aminotransferase (ALT), alkaline phosphatase (ALP), aspartate aminotransferase (AST) and gamma-glutamyl transpeptidase (GGT).

In patients with histologically proven NAFLD, serum hepatic aminotransferases, specifically alanine aminotransferase (ALT), levels are elevated from the upper limit of normal to 10 times this level (Schwimmer et al., J. Pediatr., 143(4):500-5, 2003; Rashid et al., J. Pediatr. Gastroenterol. Nutr., 30(1):48-53, 2000). The ratio of ALT/AST (aspartate aminotransferase) is >1 (range 1.5-1.7) which differs from alcoholic steatohepatitis where the ratio is generally <1. Other abnormal serologic tests that may be abnormally elevated in NASH include gamma-glutamyltransferase (gamma-GT) and fasting levels of plasma insulin, cholesterol and triglyceride.

ALT levels have been shown to be indicative of liver function. For example, normal ALT levels are about 7 to 55 units (e.g., 10-40 units) per liter correlate with normal liver function. This value is somewhat varied in children and adolescents. Thus, in some instances ALT levels less than 25 units per liter are “normal” in children and adolescents. Increased levels of ALT have been shown to correlate with liver disease and disorders. For example, NAFLD and NASH subjects typically show ALT levels of between 60 to 150 units per liter (e.g., 60-145, 70-140, 80-135, 90-130, 105-125, 110-120 units per liter, or any number between any two values thereof). In some embodiment, particularly with children and adolescents, ALT levels above 25 units per liter can be indicative of NASH or NAFLD. In determining if a subject has NAFLD or NASH or is susceptible to treatment using cysteamine and/or cystamine, ALT may be measured alone, but preferably, the determination should be made in combination with one or more other markers of liver function or dysfunction. For example, a subject having ALT levels above about 80 is indicative of liver disease or dysfunction.

AST levels have been shown to be indicative of liver function. Thus, AST levels alone or in combination with ALT or other markers can be used. For example, AST levels between about 8 to 48 units (e.g., 10-40 units) per liter correlate with normal liver function. Increased levels of AST have been shown to correlate with liver disease and disorders. For example, NAFLD and NASH subjects typically show AST levels of between 40 to 100 units per liter (e.g., 45-95, 55-90, 65-85, 70-80 units per liter, or any number between any two values thereof). In determining if a subject has NAFLD or NASH or is susceptible to treatment using cysteamine and/or cystamine, AST may be measured alone, but preferably the determination should be made in combination with one or more other markers of liver function or dysfunction. For example, a subject having AST levels above about 50 is indicative of liver disease or dysfunction.

ALP levels have been shown to be indicative of liver function. Thus, ALP levels alone or in combination with one or more of AST, ALT or other markers can be used. For example, ALP levels between about 45 to 115 units (e.g., 50-110 units) per liter correlate with normal liver function. Increased levels of ALP have been shown to correlate with liver disease and disorders. For example, NAFLD and NASH subjects typically show ALP levels of between 150 to 250 units per liter (e.g., 155-245, 160-240, 165-235, 170-230, 175-225, 180-220, 185-215, 190-210, 195-200 units per liter, or any number between any two values thereof). In determining if a subject has NAFLD or NASH or is susceptible to treatment using cysteamine and/or cystamine, ALP may be measured alone, but preferably the determination should be made in combination with one or more other markers of liver function or dysfunction. For example, a subject having ALP levels above about 150 is indicative of liver disease or dysfunction.

GGT levels have been shown to be indicative of liver function. Thus, GGT levels alone or in combination with one or more of ALP, AST, ALT or other markers can be used. For example, GGT levels between about 9 to 48 units (e.g., 10-40 units) per liter correlate with normal liver function. Increased levels of GGT have been shown to correlate with liver disease and disorders. For example, NAFLD and NASH subjects typically show GGT levels of between 50 to 100 units per liter (e.g., 55-95, 60-90, 65-85, 70-80 units per liter, or any number between any two values thereof). In determining if a subject has NAFLD or NASH or is susceptible to treatment using cysteamine and/or cystamine, GGT may be measured alone, but preferably the determination should be made in combination with one or more other markers of liver function or dysfunction. For example, a subject having GGT levels above about 50 is indicative of liver disease or dysfunction.

Triglycerides levels have been shown to be indicative of liver function. Thus, triglyceride levels alone or in combination with one or more of GGT, ALP, AST, ALT or other markers can be used. For example, triglyceride levels less than about 150 mg/dL (e.g., 100-150 mg/dL) correlate with normal liver function. Increased levels of triglycerides have been shown to correlate with liver disease and disorders. For example, NAFLD and NASH subjects typically show triglyceride levels of between 150 to 200 mg/dL (e.g., 155-195, 160-190, 165-185, 170-180 mg/dL, or any number between any two values thereof). In determining if a subject has NAFLD or NASH or is susceptible to treatment using cysteamine and/or cystamine, triglycerides may be measured alone, but preferably the determination should be made in combination with one or more other markers of liver function or dysfunction. For example, a subject having triglyceride levels above about 150 mg/dl is indicative of liver disease or dysfunction.

High triglyceride levels are known to be a leading cause of various forms of inflammation. Triglycerides are the form in which fat moves through the bloodstream. Triglycerides can be metabolized by various organs, including the liver, to form phospholipids (LDLs and HDLs), cholesterol and oxidized forms thereof. Oxidized phospholipids (OxPL) including OxLDL are known inflammatory mediators and strongly correlated with cardiovascular diseases. For example, Bieghs et al. (Hepatology, 65(3):894-903, 2012) describe that the use of antibodies to oxLDL led to a reduction in hepatic inflammation.

Increased amounts of adipose tissue are associated with decreased production of adiponectin. The reduction in adiponectin may cause or may be related to increased inflammation. Data from studies in mice and humans implicate insufficient adiponectin as a major factor in the development of fatty liver and steatohepatitis. Adiponectin circulates as trimer (low molecular weight adiponectin), hexamer (medium molecular weight adiponectin) and higher order multimer (high molecular weight adiponectin) in serum and isoform-specific effects have been demonstrated. Epidemiological studies reveal that low adiponectin levels are associated with NASH. Moreover, adiponectin is believed to have a heptoprotective effect due to protective effects against oxidative damage. Normal levels of adiponectin vary by age and sex. For example, females have a higher baseline adiponectin level compared to males. A normal weight female typically has an adiponectin level of between about 8.5 and 11 μg/ml and males typically have an adiponectin level of between about 6 and 8 μg/ml. In contrast, subject with fatty liver disease, NASH and/or obesity have adiponectin levels that are about 50-90% of normal levels (e.g., decreased by 10-50% from normal, or any value there between) (see, e.g., Merl et al. Int. J. Obes. (Lond), 29(8):998-1001, 2005).

In contrast, the resistin protein is increased in NASH subjects compared to normal subjects. Human resistin is a cysteine-rich, 108-amino-acid peptide hormone with a molecular weight of 12.5 kDa. In adult humans, resistin is expressed in bone marrow. Moreover in adipocytes of subjects having a low or healthy BMI, resistin mRNA is almost undetectable. Consistent with this, body mass index (BMI) is appears to correlate with resistin concentrations in serum, and women may have higher resistin concentrations than men. Resistin mRNA expression in human peripheral mononuclear cells is increased by proinflammatory cytokines. Serum resistin is significantly elevated in both NASH and simple steatotic subjects. Hepatic resistin is significantly increased in NASH patients in both mRNA and protein levels than those in simple steatosis and normal control subjects. Because of the cysteine-rich structure of resistin changes in sulfur availability (mainly due to cysteine and glutathione) can have an effect on the protein's structure and function. As mentioned above, cysteamine and cystamine can modulated cysteine and/or glutathione levels in subjects taking cysteamine and/or cystamine.

Subjects afflicted with NAFLD or NASH tend to be in a higher percentile of weight for their age group (e.g., above the 97^{th} percentile for BMI for their age group). Treating pediatric patients at an early stage may have lifelong benefits in the management of liver function and obesity.

The disclosure provides a method of treatment for a population of subjects with liver disease associated with periportal inflammation. In one embodiment, the subject has periportal inflammation with little to no lobular inflammation. In another embodiment, the subject has a chronic liver disorder characterized by portal tract, periportal and interface hepatitis and fibrosis. In another embodiment, the subject has a disorder selected from the group consisting of NALFD with pediatric type 2 pattern, autoimmune hepatitis, primary sclerosing cholangitis, primary biliary cholangitis, chronic drug toxicity, biliary atresia and idiopathic neonatal hepatitis syndrome. In one embodiment, the subject is an adult. In another embodiment, the subject is a juvenile. In another embodiment, the subject has NAFLD. In another embodiment, the subject has NASH.

The disclosure provides a method of treating a subject suffering from periportal inflammation and/or fatty liver disease, comprising administering a therapeutically effective amount of a cysteamine and/or cystamine or a composition or formulation comprising cysteamine and/or cystamine. In one embodiment, the fatty liver disease is selected from the group consisting of non-alcoholic fatty acid liver disease (NAFLD), non-alcoholic steatohepatitis (NASH), fatty liver disease resulting from hepatitis, fatty liver disease resulting from obesity, fatty liver disease resulting from diabetes, fatty liver disease resulting from insulin resistance, fatty liver disease resulting from hypertriglyceridemia, Abetalipoproteinemia, glycogen storage diseases, Weber-Christian disease, Wolmans disease, acute fatty liver of pregnancy, and lipodystrophy.

In certain embodiments described more fully below, pediatric and juvenile patients are treated. In one embodiment, a subject having a periportal/portal liver inflammatory condition is administered cysteamine and/or cystamine in an amount to obtain about 10-80 μmol (e.g., 10, 20, 30, 40, 50, 60, 70, 80 or any value there between) of cysteamine in the plasma. In one embodiment, the dose is about 10-40 mg/kg. In another embodiment, the dose is administered 2-4 times per day at about 300 mg to 1 gram per dose. Typically, the dose is changed over time to reach the highest tolerable dose for the subject, typically between about 30-50 μmol plasma cysteamine. For example, an initial dose may provide a circulating level of about 10 μmol cysteamine, which will be adjusted up to the highest tolerable dose, typically about 40 μmol. Similarly, an initial dose may result in a circulating level of 80 μmol, which will be adjusted down to about 40 μmol. In certain embodiments, the cysteamine and/or cystamine is administered in an immediately release formulation, a delayed release formulation, and extended release formulation, an immediate and extended release formulation, or a delayed and extended release formulation. In one embodiment, the delayed and/or extended release formulation is an enterically coated cysteamine and/or cystamine composition. In certain embodiments of any of the foregoing, subjects less than 15 years of age (e.g., less than 14, 13, 12, 11, 10, 9, 8, 7, 6, 5, 4, 3, 2 or 1 years of age) and having a body mass index (BMI) above the 97^{th} percentile for their age are treated. In some embodiments, the subject has a BMI above 97^{th} percentile for the age and weighs less than 65 kg. In some embodiment, these same subjects have high triglyceride levels, low LDH, and low or low normal adiponectin levels. In still another embodiment, the subjects have high or high normal resistin levels. In various embodiments of any of the foregoing, the patient weighs less than 65 kg. In various embodiments, the patient weighs from about 35-65 kg, or from about 40-60 kg, or from about 45-55 kg, or about 35, 40, 45, 50, 55, 60 or 65 kg. In various embodiments, a patient weighing less than 65 kg receives 600 to 1200 mg/day of cysteamine and/or cystamine or an amount to obtain circulating plasma levels of cysteamine of about 10-80 μmol (typically about 30-50 and more commonly about 40 μmol). In some instances a subject may receive more than 1200 mg/day (e.g., 2000 to 3000 mg/day). In any of the foregoing embodiments, the subject has Type 2 NASH or NASH with Type 2 histological pattern. In still another embodiment of the foregoing the subject has periportal/portal inflammation of the liver. In still another of further embodiment of any of the foregoing, the subject has low adiponectin and high triglycerides characteristic of NASH. In still another embodiment of any of the foregoing, the subject has a marker (e.g., AST, ALT, GGT or other liver marker) having a level consistent with NASH as described herein.

In various embodiments, the patient weighs 65-80 kg, and may receive 750 to about 1500 mg/day of cysteamine and/or cystamine or an amount to obtain circulating plasma levels of cysteamine of about 10-80 μmol (typically about 30-50 and more commonly about 40 μmol). In any of the foregoing embodiments, the subject has Type 2 NASH. In still another embodiment of the foregoing the subject has periportal/portal inflammation of the liver. In still another of further embodiment of any of the foregoing, the subject has low adiponectin and high triglycerides characteristic of NASH.

In various embodiments, the patient weighs more than 65 kg and receives 900 to about 2000 mg/day cysteamine and/or cystamine or an amount to obtain circulating plasma levels of cysteamine of about 10-80 μmol (typically about 30-50 and more commonly about 40 μmol). In any of the foregoing embodiments, the subject has Type 2 NASH. In still another embodiment of the foregoing the subject has periportal/portal inflammation of the liver. In still another of further embodiment of any of the foregoing, the subject has low adiponectin and high triglycerides characteristic of NASH.

The subject can be an adult, adolescent or child. In various embodiments, the patient is from 2 to 7 years old, from 8 to 12 years old, or from 13 to 18 years old. In various embodiments, an adolescent is from 10 to 19 years old as described in the National Institutes of Health standards.

In various embodiments, the administration results in a decrease in NAFLD Activity Score and in some embodiments are reduction of two or more points, no worsening or improvement of fibrosis, reduction in serum aminotransferases and gammaglutamyl transpeptidase (GGT); reduction in MRI-determined hepatic fat fraction; changes to markers of oxidation and anti-oxidant status; changes in fasting insulin and glucose; an increase in circulating adiponectin levels; a decrease in circulating resistin levels; a decrease in triglyceride levels; a decrease in oxidized phospholipids; changes in weight, height, body mass index (BMI) and waist circumference; changes in the Pediatric Quality of Life score; changes to any symptoms that patient may have experienced; proportion with a change from a histological diagnosis of definite NASH or indeterminate for NASH to not NASH at end of treatment; individual histological characteristics at end of treatment compared to baseline such as steatosis (fatty liver), lobular inflammation, portal chronic inflammation, ballooning, fibrosis score and stage 1a versus 1b fibrosis; and, change in mean NAS.

In yet another embodiment, the cysteamine and/or cystamine is administered at a frequency of 4 or less times per day (e.g., one, two or three times per day). In various embodiments, the composition is a delayed or controlled release dosage form that provides increased delivery of the cysteamine or cysteamine derivative and/or cystamine or cystamine derivative to the small intestine.

The delay or controlled release form can provide a Cₘₐₓ of the cysteamine, or a biologically active metabolite thereof, that is at least about 35%, 50%, 75% or higher than the Cₘₐₓ provided by an immediate release dosage form containing the same amount of the cysteamine and/or cystamine or derivatives thereof. In another embodiment, the delay and extended release formulation provides an improved AUC compared to immediately release forms of cysteamine and/or cystamine. For example, the AUC is increased compared to an immediate release formulation. In yet another embodiment, the delayed or controlled release dosage form comprises an enteric coating that releases the cysteamine and/or cystamine when the cysteamine and/or cystamine reaches the small intestine or a region of the gastrointestinal tract of a subject in which the pH is greater than about pH 4.5. In various embodiments, the pH is between 4.5 and 6.5. In one embodiment, the pH is about 5.5 to 6.5. In one embodiment the cysteamine and/or cystamine is delivered throughout the small intestine providing an extended release in the small intestine.

In various embodiments, the enterically coated cysteamine and/or cystamine is granulated and the granulation is compressed into a tablet or filled into a capsule. In certain embodiments, the granules are enterically coated prior to compressing or loading into a tablet or capsule, respectively. Capsule materials may be either hard or soft, and are typically sealed, such as with gelatin bands or the like. Tablets and capsules for oral use will generally include one or more commonly used excipients as discussed herein.

In a further embodiment, the cysteamine and/or cystamine is formulated for delivery as a capsule. In one embodiment, the capsule comprises the cysteamine and/or cystamine and the capsule is then enterically coated. Capsule formulations are prepared using techniques known in the art.

A suitable pH-sensitive polymer for enteric coatings is one which will dissolve in intestinal environment at a higher pH level (pH greater than 4.5), such as within the small intestine and therefore permit release of the pharmacologically active substance in the regions of the small intestine and not in the upper portion of the GI tract, such as the stomach.

In various embodiments, exemplary cysteamine and/or cystamine formulations contemplated for use in the present methods include those described in International Patent Applications PCT/US2007/002325, PCT/US2014/042607 and PCT/US2014/042616 (the disclosure of which are incorporated herein by reference).

For administration of the dosage form, i.e., the tablet or capsule comprising the enterically coated cysteamine and/or cystamine composition, a total weight in the range of approximately 50 mg to 1000 mg is used. In various embodiments, the tablet or capsule comprises 25, 50, 75, 100, 125, 150, 175, 200, 225, 250, 275, 300, 400 or 500 mg active ingredient, and multiple tablets or capsules are administered to reach the desired dosage. The dosage form is orally administered to a subject in need thereof. In another embodiment, the dosage can comprise both an immediate release and an extended release formulation, or an immediate release and a delayed release formulation, or an immediate release and an extended and delayed release formulation.

In addition, various prodrugs can be “activated” by use of the enterically coated cysteamine and/or cystamine composition. Prodrugs are pharmacologically inert, they themselves do not work in the body, but once they have been absorbed, the prodrug decomposes. The prodrug approach has been used successfully in a number of therapeutic areas including antibiotics, antihistamines and ulcer treatments. The advantage of using prodrugs is that the active agent is chemically camouflaged and no active agent is released until the drug has passed out of the gut and into the cells of the body. For example, a number of prodrugs use S—S bonds. Weak reducing agents, such as cysteamine, reduce these bonds and release the drug. Accordingly, the compositions of the disclosure are useful in combination with pro-drugs for timed release of the drug. In this aspect, a pro-drug can be administered followed by administration of an enterically coated cysteamine and/or cystamine composition of the disclosure (at a desired time) to activate the pro-drug.

In one embodiment, a cysteamine and/or cystamine formulation of the disclosure provides a delayed and extended release composition comprising cysteamine and/or cystamine (e.g., cystamine-diHCl and/or cysteamine bitartrate or other salts of cysteamine). In one embodiment, the composition comprises a core of cysteamine or salt thereof or cystamine of salt thereof and one or more pharmaceutical excipients or binder encapsulated in an enteric coating material. In one embodiment, the delayed and extended release composition comprises a core tablet of cysteamine and/or cystamine and a binder. In one embodiment, the cysteamine base of the tablet is about 50 mg and encapsulated in an enteric coating material having a thickness of about 60-100 μm (e.g., about 71, 73, 75, 77, or 79 μm or any value there between) and/or about 10-13% (e.g., about 10.5, 11.0, 11.2, 11.4, 11.6, 11.8, 12.0, 12.2, 12.4, 12.6, 12.8% of any value there between) by weight of the tablet. In another embodiment, the cysteamine base of the tablet is about 150 mg and encapsulated in an enteric coating material having a thickness of about 90-130 μm (e.g., about 97, 99, 101, 103, 105, 107, 109, 111, 113 μm or any value there between) and/or about 9-14% (e.g., about 9.5, 9.7, 9.9, 10.1, 10.3 10.5, 11.0, 11.2, 11.4, 11.6, 11.8, 12.0, 12.2, 12.4, 12.6, 12.8, 13.0, 13.2, 13.4, 13.6, 13.8% or any value there between) by weight of the tablet by weight of the tablet. In another embodiment the cysteamine base may include trace amounts of cystamine.

Cysteamine and/or cystamine compositions can be enterically coated. An enterically coated drug or tablet refers, generally, to a drug or tablet that is coated with a substance (an “enteric coating”) that remains intact or substantially intact such that the drug or tablet is passed through the stomach but dissolves and releases the drug in the small intestine.

In any of the foregoing embodiments, the enteric coating material can be selected from the group comprising polymerized gelatin, shellac, methacrylic acid copolymer type C NF, cellulose butyrate phthalate, cellulose hydrogen phthalate, cellulose proprionate phthalate, polyvinyl acetate phthalate (PVAP), cellulose acetate phthalate (CAP), cellulose acetate trimellitate (CAT), hydroxypropyl methylcellulose phthalate, hydroxypropyl methylcellulose acetate, dioxypropyl methylcellulose succinate, carboxymethyl ethylcellulose (CMEC), hydroxypropyl methylcellulose acetate succinate (HPMCAS), and acrylic acid polymers and copolymers, typically formed from methyl acrylate, ethyl acrylate, methyl methacrylate and/or ethyl methacrylate with copolymers of acrylic and methacrylic acid esters. Such an enteric formulated composition can be administered orally or parenterally.

An enteric coating can be a polymer material or materials which encase a medicament core (e.g., cystamine, cysteamine, CYSTAGON® or other cysteamine product). PROCYSBI® is an example of an enteric formulation of cysteamine. Typically a substantial amount or all of the enteric coating material is dissolved before the medicament or therapeutically active agent is released from the dosage form, so as to achieve delayed dissolution or delivery of the medicament core. A suitable pH-sensitive polymer is one which will dissolve in intestinal environment at a higher pH level (pH greater than 4.5), such as within the small intestine and therefore permit release of the pharmacologically active substance in the regions of the small intestine and not in the upper portion of the GI tract, such as the stomach.

In another embodiment, the method results in improvement in liver fibrosis compared to levels before administration of the cysteamine and/or cystamine. In yet another embodiment, the method results in a reduction in fat content of liver, a reduction in the incidence of or progression of cirrhosis, or a reduction in the incidence of hepatocellular carcinoma. In one embodiment, the method results in a decrease in hepatic aminotransferase levels compared to levels before administration of the cysteamine and/or cystamine. In a further embodiment, the administering results in a reduction in hepatic transaminase of between approximately 10% to 70%, e.g. 10, 20, 30, 40, 50, 60 or 70% or any value between these numbers, compared to levels before treatment with cysteamine and/or cystamine. In yet another embodiment, the administering results in a reduction in alanine or aspartate aminotransferase levels in a treated patient to approximately 50%, 40%, 30%, 20% or 10% above normal ALT levels, or at normal ALT levels. In yet other embodiment, the administering results in a reduction in serum ferritin levels compared to levels before treatment with the cysteamine and/or cystamine. In various embodiments, the administration results in a lowering of NAS score.

The methods, compositions and formulations of the disclosure can also include administering a second agent in combination with a cysteamine and/or cystamine ingredient to treat fatty liver disease. Thus, in another embodiment of any of the foregoing methods or compositions, the subject can be treated with a combination of active agents for treating NASH. The combination includes cysteamine and/or cystamine and one or more of metformin, statins, anti-oxidants, and/or antibodies against oxidized phospholipids. Such a combination has unexpected synergy due to a multifaceted approach to modulating inflammation and inflammatory mediators. Such a combination would increase the anti-oxidant effects of adiponectin by increasing adiponectin levels, reduce triglyceride levels thereby reducing circulating phospholipids, reduce insulin resistance, and block the proinflammatory effects of oxidized phospholipids.

The methods and compositions of the disclosure also include use of a cysteamine and/or cystamine in preparation of a medicament for treatment of fatty liver disease, and use of a cysteamine and/or cystamine in preparation of a medicament for administration in combination with a second agent for treating fatty liver disease. Also included are use of a second agent for treating fatty liver disease in preparation of a medicament for administration in combination with cysteamine and/or cystamine. Further provided are kits comprising cysteamine and/or cystamine for treatment of fatty liver disease, optionally with a second agent for treating fatty liver disease, and instructions for use in treatment of fatty liver disease. The term “fatty liver disease” may include or exclude NASH.

In various embodiments, cysteamine can include various salts. For example, salts include hydrochloride salt and bitartrate salts. In the same or a further embodiment, 1-15% of cystamine is present in combination with cysteamine.

In various embodiments of the disclosure, the cysteamine and/or cystamine, is administered at a daily dose ranging from about 10 mg/kg to about 2.5 g/kg, or from about 100 mg/kg to about 250 mg/kg, or from about 60 mg/kg to about 100 mg/kg or from about 50 mg/kg to about 90 mg/kg, or from about 30 mg/kg to about 80 mg/kg, or from about 20 mg/kg to about 60 mg/kg, or from about 10 mg/kg to about 50 mg/kg. Further, the effective dose may be 0.5 mg/kg, 1 mg/kg, 5 mg/kg, 10 mg/kg, 15 mg/kg, 20 mg/kg/25 mg/kg, 30 mg/kg, 35 mg/kg, 40 mg/kg, 45 mg/kg, 50 mg/kg, 55 mg/kg, 60 mg/kg, 70 mg/kg, 75 mg/kg, 80 mg/kg, 90 mg/kg, 100 mg/kg, 125 mg/kg, 150 mg/kg, 175 mg/kg, 200 mg/kg, 225 mg/kg, 250 mg/kg, 275 mg/kg, 300 mg/kg, 325 mg/kg, 350 mg/kg, 375 mg/kg, 400 mg/kg, 425 mg/kg, 450 mg/kg, 475 mg/kg, 500 mg/kg, 525 mg/kg, 550 mg/kg, 575 mg/kg, 600 mg/kg, 625 mg/kg, 650 mg/kg, 675 mg/kg, 700 mg/kg, 725 mg/kg, 750 mg/kg, 775 mg/kg, 800 mg/kg, 825 mg/kg, 850 mg/kg, 875 mg/kg, 900 mg/kg, 925 mg/kg, 950 mg/kg, 975 mg/kg or 1000 mg/kg, or may range between any two of the foregoing values. In some embodiments, the cysteamine and/or cystamine is administered at a total daily dose of from approximately 0.25 g/m² to 4.0 g/m² body surface area, about 0.5-2.0 g/m² body surface area, or 1-1.5 g/m² body surface area, or 1-1.95 g/m² body surface area, or 0.5-1 g/m² body surface area, or about 0.7-0.8 g/m² body surface area, or about 1.35 g/m² body surface area, or about 1.3 to about 1.95 grams/m²/day, or about 0.5 to about 1.5 grams/m²/day, or about 0.5 to about 1.0 grams/m²/day, e.g., at least about 0.3, 0.4, 0.5, 0.6, 0.7, 0.8, 0.9, 1.0, 1.1, 1.2, 1.3, 1.4, 1.5, 1.6, 1.7, 1.8, 1.9 or 2 g/m², or up to about 0.8, 0.9, 1.0, 1.1, 1.2, 1.3, 1.4, 1.5, 1.6, 1.7, 1.8, 1.9, 2.0, 2.2, 2.5, 2.7, 3.0, 3.25, 3.5 or 3.75 g/m² or may range between any two of the foregoing values.

In some embodiments, the delayed and extended release formulation comprises an enteric coating that releases the cysteamine and/or cystamine when the formulation reaches the small intestine or a region of the gastrointestinal tract of a subject in which the pH is greater than about pH 4.5. In various embodiments, the formulation releases at a pH of about 4.5 to 6.5, 4.5 to 5.5, 5.5 to 6.5 or about pH 4.5, 5.0, 5.5, 6.0 or 6.5.

The disclosure provides cysteamine and/or cystamine useful in the treatment of fatty liver diseases and disorders. A cysteamine and/or cystamine refers, generally, to cysteamine, cystamine, salts of either of the foregoing or a biologically active metabolite thereof, or combination of cysteamine and cystamine, and includes cysteamine and/or cystamine salts, esters, amides, alkylated compounds, prodrugs, analogs, phosphorylated compounds, sulfated compounds, or other chemically modified forms thereof, by such techniques as labeling (e.g., with radionuclides or various enzymes), or covalent polymer attachment such as pegylation (derivatization with polyethylene glycol).

A cysteamine and/or cystamine composition includes cysteamine, cystamine, biologically active metabolites, chemically modified forms of the compound, by such techniques as esterification, alkylation (e.g., C1, C2 or C3), labeling (e.g., with radionuclides or various enzymes), covalent polymer attachment such as pegylation (derivatization with polyethylene glycol) or any mixtures thereof. In some embodiments, cysteamine and/or cystamine compositions include, but are not limited to, hydrochloride salts, bitartrate salts, phosphorylated derivatives, and sulfated derivatives. Examples of other cysteamine and/or cystamine compositions include 2-aminopropane thiol-1, 1-aminopropane thiol-2, N- and S-substituted cysteamine, AET, aminoalkyl derivatives, phosphorothioate, amifostine (U.S. Pat. No. 4,816,482). In one embodiment, a cysteamine and/or cystamine composition specifically excludes N-acetylcysteine. In one embodiment, cysteamine and/or cystamine compositions comprise, but are not limited to, structures described below:

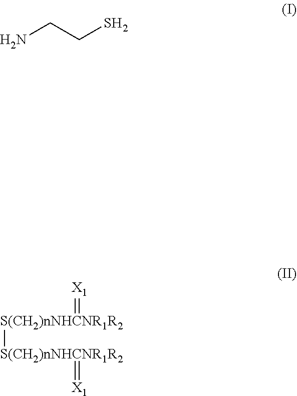

wherein n represents 2 or 3, R₁ and R₂ each represents a hydrogen atom, or an alkyl group optionally substituted by a hydroxy, amino, alkylamino or dialkylamino group, or represents a cycloalkyl or aryl group, and X₁ represents a group selected from the group consisting of ═N—CN, ═N—NO₂, ═N—COR₃, ═N—NR—COOR₃, ═N—NR—CONH₂, ═N—SO₂R₃, ═CH—NO₂, —CH—SO₂R₃, ═C(CN)₂, ═C(CN)COOR₃ and ═C(CN)CONH₂, wherein R₃ is an alkyl or aryl group. In another aspect, a cysteamine and/or cystamine composition can comprise a cysteamine radical linked to any number of non-toxic groups as set forth below:

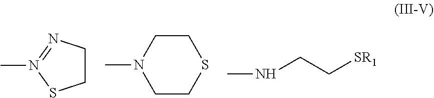

wherein R₁ represents hydrogen atom or a straight chain or a branched alkyl group having 1 to 10 carbon atoms.

Pharmaceutically acceptable salts of cysteamine or cystamine compositions are also included and comprise pharmaceutically-acceptable anions and/or cations. Pharmaceutically-acceptable cations include among others, alkali metal cations (e.g., Li⁺, Na⁺, K⁺, alkaline earth metal cations (e.g., Ca²⁺, Mg²⁺), non-toxic heavy metal cations and ammonium (NH⁴⁺) and substituted ammonium (N(R′)⁴⁺, where R′ is hydrogen, alkyl, or substituted alkyl, i.e., including, methyl, ethyl, or hydroxyethyl, specifically, trimethyl ammonium, triethyl ammonium, and triethanol ammonium cations). Pharmaceutically-acceptable anions include among other halides (e.g., Cl⁻, Br⁻), sulfate, acetates (e.g., acetate, trifluoroacetate), ascorbates, aspartates, benzoates, citrates, and lactate.

The cysteamine and/or cystamine composition may also include additional pharmaceutically acceptable carriers or vehicles. A pharmaceutically acceptable carrier or vehicle refers, generally, to materials that are suitable for administration to a subject wherein the carrier or vehicle is not biologically harmful, or otherwise, cause undesirable effects. Such carriers or vehicles are typically inert ingredients of a medicament. Typically a carrier or vehicle is administered to a subject along with an active ingredient without causing any undesirable biological effects or interacting in a deleterious manner with any of the other components of a pharmaceutical composition in which it is contained.

A cysteamine and/or cystamine composition or other active ingredient can comprise a pharmaceutically acceptable salt, ester or other derivative. For example, salts, esters or other derivatives comprise biologically active forms having a similar biological effect compared to a parent compound. Exemplary salts include hydrochloride salt and bitartrate salts.

An active ingredient, pharmaceutical or other composition of the disclosure can comprise a stabilizing agent. Stabilizing agents, generally, refer to compounds that lower the rate at which a pharmaceutical degrades, particularly an oral pharmaceutical formulation under environmental conditions of storage.

As used herein, a “therapeutically effective amount” or “effective amount” refers to that amount of the compound sufficient to result in amelioration of symptoms, for example, treatment, healing, prevention or amelioration of the relevant medical condition, or an increase in rate of treatment, healing, prevention or amelioration of such conditions, typically providing a statistically significant improvement in the treated patient population. When referencing an individual active ingredient, administered alone, a therapeutically effective dose refers to that ingredient alone. When referring to a combination, a therapeutically effective dose refers to combined amounts of the active ingredients that result in the therapeutic effect, whether administered in combination, including serially or simultaneously. In one embodiment, a therapeutically effective amount of the cysteamine and/or cystamine ameliorates symptoms, including but not limited to, liver fibrosis, fat content of liver, incidence of or progression of cirrhosis, incidence of hepatocellular carcinoma, increased hepatic aminotransferase levels, such as ALT and AST, increased serum ferritin, elevated levels of gamma-glutamyltransferase (gamma-GT), and elevated levels of plasma insulin, cholesterol and triglyceride.

The methods and compositions of the disclosure further provide enteric-coated compositions that result in less frequent dosing (2×/day vs. 4×/day), increased patient compliance and fewer gastrointestinal side effects (e.g., pain, heartburn, acid production, vomiting) and other side effects (e.g., patients smell like rotten eggs—a particular compliance problem as subjects reach puberty). The disclosure provides enteric-coated cysteamine compositions (sulfhydryl/CYSTAGON™) and cystamine compositions. Such compositions can have trace or more amounts of cystamine.

The disclosure provides methods for the treatment of fatty acid liver disease, including, but not limited to, non-alcoholic fatty acid liver disease (NAFLD), non-alcoholic steatohepatitis (NASH), fatty liver disease resulting from hepatitis, fatty liver disease resulting from obesity, fatty liver disease resulting from diabetes, fatty liver disease resulting from insulin resistance, fatty liver disease resulting from hypertriglyceridemia, Abetalipoproteinemia, glycogen storage diseases, Weber-Christian disease, Wolmans disease, acute fatty liver of pregnancy, and lipodystrophy.

The effectiveness of a method or composition of the described herein can be assessed, for example, by measuring leukocyte cystine concentrations. Additional measures of the efficacy of the methods of the disclosure include assessing relief of symptoms associated with fatty liver disease including, but not limited to, liver fibrosis, fat content of liver, incidence of or progression of cirrhosis, incidence of hepatocellular carcinoma, elevated hepatic aminotransferase levels, increased or decreased alanine aminotransferase (ALT), increased or decreased aspartate aminotransferase (AST), and elevated serum ferritin. Dosage adjustment and therapy can be made by a medical specialist depending upon, for example, the severity of fatty liver disease and/or the concentration of cystine. For example, treatment of fatty liver disease may result in a reduction in hepatic transaminase of between approximately 10% to 40% compared to levels before treatment. In a related embodiment, treatment results in a reduction in alanine aminotransferase levels in a treated patient to approximately 30%, 20% or 10% above normal ALT levels, or at normal ALT levels (≥40 IU/L). In another embodiment, treatment with cysteamine and/or cystamine results in a reduction in aspartate aminotransferase levels in a patient to approximately 30%, 20% or 10% above normal AST levels or back to normal AST levels.

In one embodiment, the disclosure provides methods of treating NAFLD using cysteamine and/or cystamine through reducing the oxidative stress caused by reactive oxygen species (ROS) in steatohepatitis. Cysteamine can achieve this through its direct or indirect ability to enhance glutathione levels within the liver. Glutathione has a protective effect against oxidative damage but itself does not enter easily into cells, even when given in large amounts treatment. Precursors of glutathione do, however, enter into cells and include cysteine, N-acetylcyteine, s-adenosylmethionine (SAMe) and other sulphur-containing compounds such as cysteamine.

The compositions of the disclosure can be used in combination with a second agent or other therapies useful for treating NAFLD or NASH or other fatty acid liver disorders. For example, cysteamine and/or cystamine may be administered with drugs such as glitazones/thiazolidinediones that combat insulin resistance, including mesylate (troglitazone (REZULIN®)), rosiglitazone (AVANDIA®), pioglitazone (ACTOS®), as well as other agents, including, but not limited to, metformin, Sulfonylureas, Alpha-glucosidase inhibitors, Meglitinides, vitamin E, tetrahydrolipstatin (XENICAL™, milk thistle protein (SILIPHOS®), and anti-virals.

Other therapies which reduce side effects of cysteamine and/or cystamine can be combined with the methods and compositions of the disclosure to treat diseases and disorders that are attributed or result from NAFLD or NASH. Urinary phosphorus loss, for example, entails rickets, and it may be necessary to give a phosphorus supplement. Carnitine is lost in the urine and blood levels are low. Carnitine allows fat to be used by the muscles to provide energy. Hormone supplementation is sometimes necessary. Sometimes the thyroid gland will not produce enough thyroid hormones. This is given as thyroxin (drops or tablets). Insulin treatment is sometimes necessary if diabetes appears, when the pancreas does not produce enough insulin. These treatments have become rarely necessary in children whom are treated with cysteamine and/or cystamine, since the treatment protects the thyroid and the pancreas. Some adolescent boys require a testosterone treatment if puberty is late. Growth hormone therapy may be indicated if growth is not sufficient despite a good hydro electrolytes balance. Accordingly, such therapies can be combined with the cysteamine and/or cystamine compositions and methods of the disclosure. Additional therapies including the use of omeprazole (PRILOSEC®) can reduce adverse symptoms affecting the digestive tract.

The disclosure provides cysteamine and/or cystamine useful in the treatment of fatty liver diseases and disorders. To administer cysteamine and/or cystamine of the disclosure to human or test animals, it is useful to formulate the cysteamine and/or cystamine compositions in a composition comprising one or more pharmaceutically acceptable carriers. As set out above, pharmaceutically or pharmacologically acceptable carriers or vehicles refer to molecular entities and compositions that do not produce allergic, or other adverse reactions when administered using routes well-known in the art, as described below, or are approved by the U.S. Food and Drug Administration or a counterpart foreign regulatory authority as an acceptable additive to orally or parenterally administered pharmaceuticals. Pharmaceutically acceptable carriers include any and all clinically useful solvents, dispersion media, coatings, antibacterial and antifungal agents, isotonic and absorption delaying agents and the like.

Pharmaceutical carriers include pharmaceutically acceptable salts, particularly where a basic or acidic group is present in a compound. For example, when an acidic substituent, such as —COOH, is present, the ammonium, sodium, potassium, calcium and the like salts, are contemplated for administration. Additionally, where an acid group is present, pharmaceutically acceptable esters of the compound (e.g., methyl, tert-butyl, pivaloyloxymethyl, succinyl, and the like) are contemplated forms of the compounds, such esters being known in the art for modifying solubility and/or hydrolysis characteristics for use as sustained release or prodrug formulations.

When a basic group (such as amino or a basic heteroaryl radical, such as pyridyl) is present, then an acidic salt, such as hydrochloride, hydrobromide, acetate, maleate, pamoate, phosphate, methanesulfonate, p-toluenesulfonate, and the like, is contemplated as a form for administration.

In addition, compounds may form solvates with water or common organic solvents. Such solvates are contemplated as well.

The cysteamine and/or cystamine and compositions thereof may be administered orally, parenterally, transocularly, intranasally, transdermally, transmucosally, by inhalation spray, vaginally, rectally, or by intracranial injection. The term parenteral as used herein includes subcutaneous injections, intravenous, intramuscular, intracisternal injection, or infusion techniques. Administration by intravenous, intradermal, intramusclar, intramammary, intraperitoneal, intrathecal, retrobulbar, intrapulmonary injection and or surgical implantation at a particular site is contemplated as well. Generally, compositions for administration by any of the above methods are essentially free of pyrogens, as well as other impurities that could be harmful to the recipient. Further, compositions for administration parenterally are sterile.

Pharmaceutical compositions of the disclosure containing a cysteamine and/or cystamine as an active ingredient may contain pharmaceutically acceptable carriers or additives depending on the route of administration. Examples of such carriers or additives include water, a pharmaceutically acceptable organic solvent, collagen, polyvinyl alcohol, polyvinylpyrrolidone, a carboxyvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene glycol, Vaseline, paraffin, stearyl alcohol, stearic acid, human serum albumin (HSA), mannitol, sorbitol, lactose, a pharmaceutically acceptable surfactant and the like. Additives used are chosen from, but not limited to, the above or combinations thereof, as appropriate, depending on the dosage form of the disclosure.

Formulation of the pharmaceutical composition will vary according to the route of administration selected (e.g., solution, emulsion). An appropriate composition comprising the cysteamine and/or cystamine to be administered can be prepared in a physiologically acceptable vehicle or carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers.

A variety of aqueous carriers, e.g., water, buffered water, 0.4% saline, 0.3% glycine, or aqueous suspensions may contain the active compound in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

In some embodiments, the cysteamine and/or cystamine can be lyophilized for storage and reconstituted in a suitable carrier prior to use. Any suitable lyophilization and reconstitution techniques can be employed. It is appreciated by those skilled in the art that lyophilization and reconstitution can lead to varying degrees of activity loss and that use levels may have to be adjusted to compensate.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

In one embodiment, the disclosure provides use of an enterically coated cysteamine and/or cystamine composition. Enteric coatings prolong release until the cysteamine and/or cystamine composition reaches the intestinal tract, typically the small intestine. Because of the enteric coatings, delivery to the small intestine is improved thereby improving uptake of the active ingredient while reducing gastric side effects.

In some embodiments, the coating material is selected such that the therapeutically active agent is released when the dosage form reaches the small intestine or a region in which the pH is greater than pH 4.5. The coating may be a pH-sensitive material, which remain intact in the lower pH environs of the stomach, but which disintegrate or dissolve at the pH commonly found in the small intestine of the patient. For example, the enteric coating material begins to dissolve in an aqueous solution at pH between about 4.5 to about 5.5. For example, pH-sensitive materials will not undergo significant dissolution until the dosage from has emptied from the stomach. The pH of the small intestine gradually increases from about 4.5 to about 6.5 in the duodenal bulb to about 7.2 in the distal portions of the small intestine. In order to provide predictable dissolution corresponding to the small intestine transit time of about 3 hours (e.g., 2-3 hours) and permit reproducible release therein, the coating should begin to dissolve at the pH range within the small intestine. Therefore, the amount of enteric polymer coating should be sufficient to substantially dissolved during the approximate three hour transit time within the small intestine, such as the proximal and mid-intestine.

Enteric coatings have been used to arrest the release of the drug from orally ingestible dosage forms. Depending upon the composition and/or thickness, the enteric coatings are resistant to stomach acid for required periods of time before they begin to disintegrate and permit release of the drug in the lower stomach or upper part of the small intestines. Examples of some enteric coatings are disclosed in U.S. Pat. No. 5,225,202 which is incorporated by reference fully herein. As set forth in U.S. Pat. No. 5,225,202, some examples of coating previously employed are beeswax and glyceryl monostearate; beeswax, shellac and cellulose; and cetyl alcohol, mastic and shellac, as well as shellac and stearic acid (U.S. Pat. No. 2,809,918); polyvinyl acetate and ethyl cellulose (U.S. Pat. No. 3,835,221); and neutral copolymer of polymethacrylic acid esters (Eudragit L30D) (F. W. Goodhart et al., Pharm. Tech., pp. 64-71, April 1984); copolymers of methacrylic acid and methacrylic acid methylester (Eudragits), or a neutral copolymer of polymethacrylic acid esters containing metallic stearates (Mehta et al., U.S. Pat. Nos. 4,728,512 and 4,794,001). Such coatings comprise mixtures of fats and fatty acids, shellac and shellac derivatives and the cellulose acid phthlates, e.g., those having a free carboxyl content. See, Remington's at page 1590, and Zeitova et al. (U.S. Pat. No. 4,432,966), for descriptions of suitable enteric coating compositions. Accordingly, increased adsorption in the small intestine due to enteric coatings of cysteamine and/or cystamine compositions can result in improved efficacy.

Generally, the enteric coating comprises a polymeric material that prevents cysteamine and/or cystamine release in the low pH environment of the stomach but that ionizes at a slightly higher pH, typically a pH of 4 or 5, and thus dissolves sufficiently in the small intestines to gradually release the active agent therein. Accordingly, among the most effective enteric coating materials are polyacids having a pKa in the range of about 3 to 5. Suitable enteric coating materials include, but are not limited to, polymerized gelatin, shellac, methacrylic acid copolymer type C NF, cellulose butyrate phthalate, cellulose hydrogen phthalate, cellulose proprionate phthalate, polyvinyl acetate phthalate (PVAP), cellulose acetate phthalate (CAP), cellulose acetate trimellitate (CAT), hydroxypropyl methylcellulose phthalate, hydroxypropyl methylcellulose acetate, dioxypropyl methylcellulose succinate, carboxymethyl ethylcellulose (CMEC), hydroxypropyl methylcellulose acetate succinate (HPMCAS), and acrylic acid polymers and copolymers, typically formed from methyl acrylate, ethyl acrylate, methyl methacrylate and/or ethyl methacrylate with copolymers of acrylic and methacrylic acid esters (Eudragit NE, Eudragit RL, Eudragit RS). For example, the enterically coating can comprise Eudragit L30D, triethylcitrate, and hydroxypropylmethylcellulose (HPMC), wherein the coating comprises 10 to 13% of the final product.

In one embodiment, the cysteamine and/or cystamine composition is administered in tablet form. In one embodiment, the tablets are manufactured by first enterically coating the cysteamine and/or cystamine. A method for forming tablets herein is by direct compression of the powders containing the enterically coated cysteamine and/or cystamine, optionally in combination with diluents, binders, lubricants, disintegrants, colorants, stabilizers or the like. As an alternative to direct compression, compressed tablets can be prepared using wet-granulation or dry-granulation processes. Tablets may also be molded rather than compressed, starting with a moist material containing a suitable water-soluble lubricant.

In various embodiments, the enterically coated cysteamine and/or cystamine is granulated and the granulation is compressed into a tablet or filled into a capsule. In certain embodiments, the granules are enterically coated prior to compressing into a tablet or capsule. Capsule materials may be either hard or soft, and are typically sealed, such as with gelatin bands or the like. Tablets and capsules for oral use will generally include one or more commonly used excipients as discussed herein.

In a further embodiment, the cysteamine and/or cystamine is formulated as a capsule. In one embodiment, the capsule comprises the cysteamine and/or cystamine and the capsule is then enterically coated. Capsule formulations are prepared using techniques known in the art.

A suitable pH-sensitive polymer is one which will dissolve in intestinal environment at a higher pH level (pH greater than 4.5), such as within the small intestine and therefore permit release of the pharmacologically active substance in the regions of the small intestine and not in the upper portion of the GI tract, such as the stomach.

In various embodiments, exemplary cysteamine and/or cystamine formulations contemplated for use in the methods are described in International Patent Applications PCT/US14/42607 and PCT/US14/42616.

For administration of the dosage form, i.e., the tablet or capsule comprising the enterically coated cysteamine and/or cystamine, a total weight in the range of approximately 100 mg to 1000 mg is used. In various embodiments, the tablet or capsule comprises 25, 50, 75, 100, 125, 150, 175, 200, 225, 250, 275, 300, 400 or 500 mg active ingredient, and multiple tablets or capsules are administered to reach the desired dosage. The dosage form is orally administered to a subject need thereof.

In some embodiments, the cysteamine and/or cystamine composition is a delayed or controlled release dosage form that provides a Cₘₐₓ of the cysteamine and/or cystamine product that is at least about 35%, 40%, 45%, 50%, 55%, 60%, 65%, 70%, 75%, 80%, 85%, or 100% higher than the Cₘₐₓ provided by an immediate release dosage form containing the same amount of the cysteamine and/or cystamine. In some embodiments, the Cₘₐₓ is up to about 75%, 100%, 125% or 150% higher than the Cₘₐₓ of the immediate release dosage form. Cₘₐₓ refers to the maximum dose of the cysteamine and/or cystamine in the blood after dosing and provides an indicator that the drug is absorbed systemically.

In some embodiments, the AUC of the delayed or controlled release dosage form is also increased by at least about 20%, 25%, 30%, 35%, 40%, 45%, or 50%, or up to about 50%, 60%, 75% or 100% relative to an immediate release dosage form. AUC or “area under the curve”, and refers to the kinetic curve derived when plasma drug concentration versus time is measured after dosing of a drug.

The preparation of delayed, controlled or sustained/extended release forms of pharmaceutical compositions with the desired pharmacokinetic characteristics can be accomplished by a variety of methods. For example, oral controlled delivery systems include dissolution-controlled release (e.g., encapsulation dissolution control or matrix dissolution control), diffusion-controlled release (reservoir devices or matrix devices), ion exchange resins, osmotic controlled release or gastroretentive systems. Dissolution controlled release can be obtained, e.g., by slowing the dissolution rate of a drug in the gastrointestinal tract, incorporating the drug in an in soluble polymer, and coating drug particles or granules with polymeric materials of varying thickness. Diffusion controlled release can be obtained, e.g., by controlling diffusion through a polymeric membrane or a polymeric matrix. Osmotically controlled release can be obtained, e.g., by controlling solvent influx across a semipermeable membrane, which in turn carries the drug outside through a laser-drilled orifice. The osmotic and hydrostatic pressure differences on either side of the membrane govern fluid transport. Prolonged gastric retention may be achieved by, e.g., altering density of the formulations, bioadhesion to the stomach lining, or increasing floating time in the stomach. For further detail, see the Handbook of Pharmaceutical Controlled Release Technology, Wise, ed., Marcel Dekker, Inc., New York, N.Y. (2000), incorporated by reference herein in its entirety, e.g. Chapter 22 (“An Overview of Controlled Release Systems”).

The concentration of cysteamine and/or cystamine in these formulations can vary widely, for example from less than about 0.5%, usually at or at least about 1% to as much as 15 or 20% by weight and are selected primarily based on fluid volumes, manufacturing characteristics, viscosities, etc., in accordance with the particular mode of administration selected. Actual methods for preparing administrable compositions are known or apparent to those skilled in the art and are described in more detail in, for example, Remington's Pharmaceutical Science, 15th ed., Mack Publishing Company, Easton, Pa. (1980).

The cysteamine and/or cystamine is present in the composition in a therapeutically effective amount; typically, the composition is in unit dosage form. The amount of cysteamine and/or cystamine composition administered will, of course, be dependent on the age, weight, and general condition of the subject, the severity of the condition being treated, and the judgment of the prescribing-physician. Suitable therapeutic amounts will be known to those skilled in the art and/or are described in the pertinent reference texts and literature. Current non-enterically coated doses are about 1.35 g/m² body surface area and are administered 4-5 times per day. In one aspect, the dose is administered either one time per day or multiple times per day. The cysteamine and/or cystamine composition may be administered one, two or three or four times per day.

In some embodiments, the cysteamine and/or cystamine composition may be administered at a total daily dose of about 1-1.5 g/m² body surface area, or 0.5-1 g/m² body surface area, or about 0.7-0.8 g/m² body surface area, or about 1.35 g/m² body surface area. Salts or esters of the same active ingredient may vary in molecular weight depending on the type and weight of the salt or ester moiety. For administration of the dosage form, e.g., a tablet or capsule or other oral dosage form comprising the enterically coated cysteamine and/or cystamine, a total weight in the range of approximately 100 mg to 1000 mg is used. The dosage form is orally administered to a patient suffering from fatty liver disease for which a cysteamine and/or cystamine would be indicated, including, but not limited to, NAFLD and NASH. Administration may continue for at least 3 months, 6 months, 9 months, 1 year, 2 years, or more.

Compositions useful for administration may be formulated with uptake or absorption enhancers to increase their efficacy. Such enhancer include for example, salicylate, glycocholate/linoleate, glycholate, aprotinin, bacitracin, SDS, caprate and the like. See, e.g., Fix (J. Pharm. Sci., 85:1282-1285, 1996) and Oliyai and Stella (Ann. Rev. Pharmacol. Toxicol., 32:521-544, 1993).

The enterically coated cysteamine and/or cystamine composition can comprise various excipients, as is well known in the pharmaceutical art, provided such excipients do not exhibit a destabilizing effect on any components in the composition. Thus, excipients such as binders, bulking agents, diluents, disintegrants, lubricants, fillers, carriers, and the like can be combined with the cysteamine and/or cystamine composition. For solid compositions, diluents are typically necessary to increase the bulk of a tablet so that a practical size is provided for compression. Suitable diluents include dicalcium phosphate, calcium sulfate, lactose, cellulose, kaolin, mannitol, sodium chloride, dry starch and powdered sugar. Binders are used to impart cohesive qualities to a tablet formulation, and thus ensure that a tablet remains intact after compression. Suitable binder materials include, but are not limited to, starch (including corn starch and pregelatinized starch), gelatin, sugars (including sucrose, glucose, dextrose and lactose), polyethylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, hydroxyethyl cellulose, and the like), and Veegum. Lubricants are used to facilitate tablet manufacture; examples of suitable lubricants include, for example, magnesium stearate, calcium stearate, and stearic acid, and are typically present at no more than approximately 1 weight percent relative to tablet weight. Disintegrants are used to facilitate tablet disintegration or “breakup” after administration, and are generally starches, clays, celluloses, algins, gums or crosslinked polymers. If desired, the pharmaceutical composition to be administered may also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, and the like. If desired, flavoring, coloring and/or sweetening agents may be added as well. Other optional components for incorporation into an oral formulation herein include, but are not limited to, preservatives, suspending agents, thickening agents, and the like. Fillers include, for example, insoluble materials such as silicon dioxide, titanium oxide, alumina, talc, kaolin, powdered cellulose, microcrystalline cellulose, and the like, as well as soluble materials such as mannitol, urea, sucrose, lactose, dextrose, sodium chloride, sorbitol, and the like.

A pharmaceutical composition may also comprise a stabilizing agent such as hydroxypropyl methylcellulose or polyvinylpyrrolidone, as disclosed in U.S. Pat. No. 4,301,146. Other stabilizing agents include, but are not limited to, cellulosic polymers such as hydroxypropyl cellulose, hydroxyethyl cellulose, methyl cellulose, ethyl cellulose, cellulose acetate, cellulose acetate phthalate, cellulose acetate trimellitate, hydroxypropyl methylcellulose phthalate, microcrystalline cellulose and carboxymethylcellulose sodium; and vinyl polymers and copolymers such as polyvinyl acetate, polyvinylacetate phthalate, vinylacetate crotonic acid copolymer, and ethylene-vinyl acetate copolymers. The stabilizing agent is present in an amount effective to provide the desired stabilizing effect; generally, this means that the ratio of cysteamine and/or cystamine composition to the stabilizing agent is at least about 1:500 w/w, more commonly about 1:99 w/w.

In an alternative embodiment, the enterically coated cysteamine and/or cystamine are granulated and the granulation is compressed into a tablet or filled into a capsule. Capsule materials may be either hard or soft, and are typically sealed, such as with gelatin bands or the like. Tablets and capsules for oral use will generally include one or more commonly used excipients as discussed herein.

For administration of the dosage form, i.e., the tablet or capsule comprising the enterically coated cysteamine and/or cystamine, a total weight in the range of approximately 100 mg to 1000 mg is used. The dosage form is orally administered to a patient suffering from a condition for which a cysteamine and/or cystamine composition would typically be indicated, including, but not limited to, NAFLD and NASH.

The compositions of the disclosure can be used in combination with other therapies useful for treating NAFL and NASH. For example, antioxidants such as glycyrrhizin, schisandra extract, ascorbic acid, glutathione, silymarin, lipoic acid, and d-alpha-tocopherol, and parenterally administering to the subject glycyrrhizin, ascorbic acid, glutathione, and vitamin B-complex may be administered in combination (either simultaneously in a single composition or in separate compositions). Alternatively, the combination of therapeutics can be administered sequentially.

It is to be understood that while the disclosure has been described in conjunction with specific embodiments thereof, that the foregoing description as well as the examples which follow are intended to illustrate and not limit the scope of the disclosure. Other aspects, advantages and modifications within the scope of the disclosure will be apparent to those skilled in the art to which the disclosure.

Numerous modifications and variations in the invention as set forth in the above illustrative examples are expected to occur to those skilled in the art. Consequently only such limitations as appear in the appended claims should be placed on the invention.

1. A method of treating a subject or patient suffering from a disease or disorder associated with periportal/portal liver inflammation comprising administering a therapeutically effective amount of a cysteamine salt and/or cystamine salt to the subject or patient to obtain a systemic plasma level of about 10-80 μmol of cysteamine in the plasma and/or wherein the inflammation is reduced and/or liver function markers are improved.

2. The method of claim 1, wherein the fatty liver disease is selected from the group consisting of NAFLD with pediatric type 2 pattern, autoimmune hepatitis, primary sclerosing cholangitis, primary biliary cholangitis, chronic drug toxicity, biliary atresia, and idiopathic neonatal hepatitis syndrome.

3. (canceled)

4. The method of claim 1, wherein the liver disease or disorder is selected from the group consisting of NAFLD with pediatric type 2 pattern, autoimmune hepatitis, primary sclerosing cholangitis, primary biliary cholangitis, chronic drug toxicity, biliary atresia, idiopathic neonatal hepatitis syndrome.

5. The method of claim 1, further comprising measuring liver function markers selected from alanine aminotransferase (ALT), alkaline phosphatase (ALP), aspartate aminotransferase (AST), gamma-glutamyl transpeptidase (GGT) and GST.

6. (canceled)

7. The method of claim 1, wherein the subject or patient is obese.

8. A method of treating a subject or patient at risk for developing a hepatic disease or disorder associated with periportal/portal inflammation or fibrosis, wherein the subject is at risk for liver disease and has normal liver function, comprising administering cysteamine salt and/or cystamine salt to the subject or patient.

9. The method of claim 5, wherein the subject or patient comprises normal liver function markers or abnormal liver function markers, wherein the markers of liver function are selected from the group consisting of alanine aminotransferase (ALT), alkaline phosphatase (ALP), aspartate aminotransferase (AST), gamma-glutamyl transpeptidase (GGT) and triglycerides.

10. The method of claim 9, wherein (i) an ALT level is normal or elevated and wherein an elevated level of about 60 or higher units/liter is indicative of fatty liver disease; (ii) wherein an ALP level is normal or elevated and wherein an elevated level of about 150 or higher units/liter is indicative of fatty liver disease; (iii) wherein an AST level is normal or elevated and wherein an elevated level of about 40 or higher units/liter is indicative of fatty liver disease; (iv) wherein a GGT level is normal or elevated and wherein an elevated level of about 50 or higher units/liter is indicative of fatty liver disease; and/or (v) wherein a triglyceride level above 150 mg/dL and/or high LDL level is indicative of fatty liver disease.

11-14. (canceled)

15. The method of claim 10, wherein the subject's or patient's BMI is above the 97^{th} percentile for the subject's or patient's age and the subject or patient weighs less than or equal to 65 kg.

16. (canceled)

17. The method of claim 8, wherein the cysteamine and/or cystamine salt composition is an enterically coated composition.

18. The method of claim 17, wherein the cysteamine salt is cysteamine bitartrate.

19. The method of claim 17, wherein the cysteamine salt compositions comprise granules or tablets.

20. The method of claim 17, wherein the cysteamine salt composition comprises (i) a core particle comprising a mixture of cysteamine bitartrate and a binder, and (ii) an enteric membrane surrounding the core particle; wherein the beads have a distribution of particle sizes in a range of about 0.7 mm to about 2.8 mm; wherein the enteric membrane begins to dissolve within a pH range of about 4.5 to about 6.5; wherein the enteric membrane is present in an amount in a range of about 25% to about 35% by weight, based on the weight of the core particles; and wherein the pharmaceutical dosage form, upon administration in a capsule to fasted healthy normal subjects at 600 mg free cysteamine base, provides: (a) a mean Cₘₐₓ upon oral dosing in a range of 2.3±0.6 mg/L or in a range of 80% to 125% thereof; (b) a mean AUC (0-∞) upon oral dosing in a range of 0.84±0.19 min*mg/L/mg or in a range of 80% to 125% thereof; and (c) a plasma cysteamine level of 10-80 μmol.

21. The method of claim 17, wherein the cysteamine salt composition comprises (i) a core tablet comprising a mixture of cysteamine bitartrate and a binder, and (ii) an enteric membrane surrounding the tablet, wherein the thickness of the enteric coating increases from 70 μm to 115 μm relative to the cysteamine base dose from 50 mg to 300 mg, and/or wherein the enteric coating is present in an amount in a range of about 9 to about 15% by weight of the core tablet and wherein upon delivery to a fasted healthy normal subject at 600 mg free cysteamine base the dose provides a plasma cysteamine level of 10-80 μm.

22. The method of claim 17, wherein the cystamine salt composition is an enterically coated composition.

23. The method of claim 22, wherein the cystamine salt compositions comprise granules or tablets.

24. The method of claim 22, wherein the cystamine salt composition comprises (i) a core particle comprising a mixture of cystamine and a binder, and (ii) an enteric membrane surrounding the core particle; wherein the beads have a distribution of particle sizes in a range of about 0.7 mm to about 2.8 mm; wherein the enteric membrane begins to dissolve within a pH range of about 4.5 to about 6.5; wherein the enteric membrane is present in an amount in a range of about 25% to about 35% by weight, based on the weight of the core particles; and wherein the pharmaceutical dosage form, upon administration in a capsule to fasted healthy normal subjects at 600 mg free cystamine base, provides: (a) a mean Cₘₐₓ upon oral dosing in a range of 2.3±0.6 mg/L or in a range of 80% to 125% thereof; (b) a mean AUC (0-∞) upon oral dosing in a range of 0.84±0.19 min*mg/L/mg or in a range of 80% to 125% thereof; and (c) a plasma cysteamine level of 10-80 μmol.

25. The method of claim 22, wherein the cystamine salt composition comprises (i) a core tablet comprising a mixture of cystamine and a binder, and (ii) an enteric membrane surrounding the core tablet, wherein the thickness of the enteric coating increases from 60 μm to 130 μm relative to the cystamine dose from 50 mg to 300 mg, and/or wherein the enteric coating is present in an amount in a range of about 9 to about 15% by weight of the core tablet and wherein upon delivery to a fasted healthy normal subject at 600 mg cystamine the dose provides a plasma cysteamine level of 10-80 μm.
